(19) United States
(12) Patent Application Publication
(10) Pub. No.: US 2026/0301430 A1
(43) Pub. Date: Oct. 1, 2026

(54) DROWSINESS ESTIMATION DEVICE AND DROWSINESS ESTIMATION METHOD

(71) Applicant: Mitsubishi Electric Corporation, Tokyo (JP)

(72) Inventor: Koki UENO, Tokyo (JP)

(73) Assignee: Mitsubishi Electric Corporation, Tokyo (JP)

(21) Appl. No.: 19/490,064

(22) PCT Filed: Jun. 29, 2023
(86) PCT No.: PCT/JP2023/024104

§ 371 Date: Dec. 4, 2025

(51) Int. Cl. G06V 20/59; G06V 10/74; G06V 40/18; G06V 40/20

(52) U.S. Cl. CPC G06V 20/597; G06V 10/761; G06V 40/18; G06V 40/20

(57) ABSTRACT

There are included: a sensing unit to acquire drowsiness related information for each of frames on the basis of frames of a captured image obtained by capturing a face of an occupant of a mobile object; a first noise factor detecting unit to detect a noise factor action by the occupant on the basis of the drowsiness related information; a feature amount calculating unit to calculate a drowsiness estimation feature amount on the basis of post-noise factor removal drowsiness related information after excluding the drowsiness related information that is a source of detection of the noise factor action; a drowsiness score calculating unit to calculate a drowsiness score using the drowsiness estimation feature amount calculated by the feature amount calculating unit; and a drowsiness estimation unit to estimate the drowsiness of the occupant on the basis of the drowsiness score calculated by the drowsiness score calculating unit.

### TECHNICAL FIELD

The present disclosure relates to a drowsiness estimation device and a drowsiness estimation method.

### BACKGROUND ART

In general, as a feature of a person when drowsiness occurs, there is a feature that a degree of opening of eyes becomes small or a number of blinks increases.

Conventionally, as a technique for estimating drowsiness of an occupant of a mobile object based on this, a technique for estimating the drowsiness using a feature amount such as an opening degree of eyes or the number of blinks extracted using face information of the occupant is known (for example, Patent Literature 1).

### CITATION LIST

### Patent Literature

Patent Literature 1: JP 2011-48531 A

### SUMMARY OF INVENTION

### Technical Problem

Action of a person includes an action having a feature similar to a feature when drowsiness occurs, such as what is called downward looking, what is called frowning, or smiling.

In the related art, when the drowsiness of the occupant is estimated, there is a problem that the estimation accuracy of the drowsiness of the occupant may decrease in a case where the occupant takes an action having a feature similar to a feature at the time of occurrence of the drowsiness as described above.

Note that, in the technique disclosed in Patent Literature 1, in a case where a face direction angle changes immediately after movement of the eyelid occurs or in a case where movement of the eyelid occurs and movement of the line of sight also occurs, there is a possibility that, in order not to perform determination of drowsiness, necessary determination of drowsiness is not performed in the first place, and a problem that the estimation accuracy of the drowsiness of the occupant is lowered is still not solved.

The present disclosure has been made to solve the above problems, and an object thereof is to provide a drowsiness estimation device that prevents, in estimating drowsiness of an occupant in a mobile object, a decrease in estimation accuracy of the drowsiness of the occupant due to that the occupant takes an action in which a feature similar to a feature at the time of occurrence of drowsiness is observed.

### Solution to Problem

A drowsiness estimation device according to the present disclosure includes: a sensing unit to acquire drowsiness related information indicating a state related to drowsiness of an occupant of a mobile object for each of frames on a basis of frames of a captured image obtained by capturing a face of the occupant; a first noise factor detecting unit to detect a noise factor action that is an action accompanied by eye movement and is similar to an action of the occupant caused by drowsiness on a basis of the drowsiness related information acquired by the sensing unit; a feature amount calculating unit to calculate a drowsiness estimation feature amount for estimating the drowsiness of the occupant on a basis of post-noise factor removal drowsiness related information after excluding the drowsiness related information that is a source of detection of the noise factor action by the first noise factor detecting unit from the drowsiness related information acquired by the sensing unit; a drowsiness score calculating unit to calculate a drowsiness score using the drowsiness estimation feature amount calculated by the feature amount calculating unit; and a drowsiness estimation unit to estimate the drowsiness of the occupant on a basis of the drowsiness score calculated by the drowsiness score calculating unit.

### Advantageous Effects of Invention

According to the present disclosure, in estimating drowsiness of an occupant of a mobile object, it is possible to prevent a decrease in estimation accuracy of the drowsiness of the occupant due to that the occupant takes an action in which a feature similar to a feature when the drowsiness occurs is observed.

### BRIEF DESCRIPTION OF DRAWINGS

FIG. 1 is a diagram illustrating a configuration example of a drowsiness estimation device according to a first embodiment.

FIG. 2 is a flowchart describing an operation of the drowsiness estimation device according to the first embodiment.

FIG. 3 is a flowchart describing details of feature amount calculation processing in a case where a feature amount calculating unit specifies post-noise factor removal drowsiness related information on the basis of an exclusion target flag attached to drowsiness related information as an exclusion target by a sensing result selecting unit, and calculates a drowsiness estimation feature amount on the basis of the specified post-noise factor removal drowsiness related information in the first embodiment.

FIG. 4 is a flowchart for describing details of feature amount calculation processing in a case where the feature amount calculating unit calculates a drowsiness estimation feature amount on the basis of the post-noise factor removal drowsiness related information output from the sensing result selecting unit in the first embodiment.

FIGS. 5A and 5B are diagrams illustrating an example of a hardware configuration of the drowsiness estimation device according to the first embodiment.

FIG. 6 is a diagram illustrating a configuration example of a drowsiness estimation device according to a second embodiment.

FIG. 7 is a flowchart for describing an operation of the drowsiness estimation device according to the second embodiment.

FIG. 8 is a diagram illustrating a configuration example of a drowsiness estimation device according to a third embodiment.

FIG. 9 is a flowchart for describing an operation of the drowsiness estimation device according to the third embodiment.

FIG. 10 is a diagram illustrating a configuration example of a drowsiness estimation device obtained by combining configurations of the drowsiness estimation device according to the first embodiment, the drowsiness estimation device according to the second embodiment, and the drowsiness estimation device according to the third embodiment.

FIG. 11 is a flowchart for describing an operation of the drowsiness estimation device obtained by combining the configurations of the drowsiness estimation device according to the first embodiment, the drowsiness estimation device according to the second embodiment, and the drowsiness estimation device according to the third embodiment.

### DESCRIPTION OF EMBODIMENTS

Hereinafter, embodiments of the present disclosure will be described in detail with reference to the drawings.

### First Embodiment

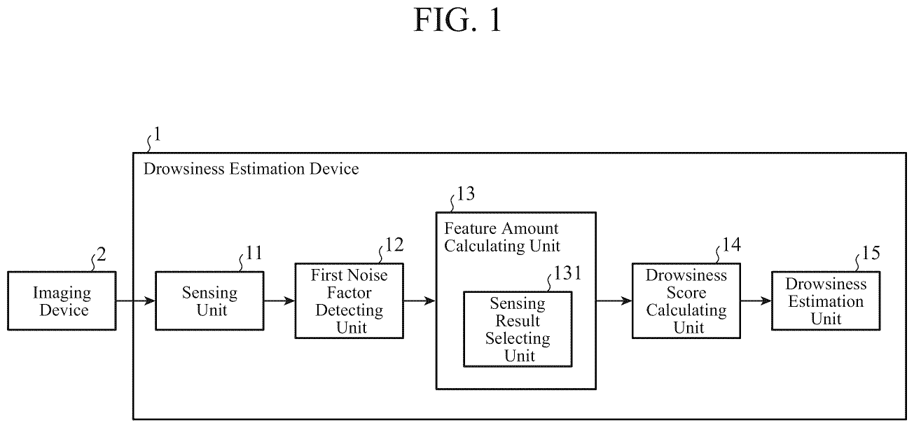

FIG. 1 is a diagram illustrating a configuration example of a drowsiness estimation device 1 according to a first embodiment.

The drowsiness estimation device 1 according to the first embodiment is connected to an imaging device 2, and estimates drowsiness of a person (hereinafter referred to as “subject”) to be estimated on the basis of an image captured by the imaging device 2.

In the first embodiment, the subject is assumed to be a driver of a vehicle (not illustrated). Further, the drowsiness estimation device 1 according to the first embodiment is assumed to be mounted on a vehicle.

In the following first embodiment, the driver of the vehicle is also simply referred to as a “driver”.

The imaging device 2 is mounted on a vehicle. The imaging device 2 is disposed in a central portion of a dashboard of a vehicle, an A-pillar, a meter panel, or the like so as to be able to image at least a face of a driver. The imaging device 2 may be shared with what is called a “Driver Monitoring System (DMS)”.

The imaging device 2 is a visible light camera or an infrared camera. In a case where the imaging device 2 is an infrared camera, the infrared camera is provided with a light source (not illustrated) that emits infrared rays for imaging to a range including the face of the driver. The light source includes, for example, a light emitting diode (LED).

The imaging device 2 outputs a captured image (hereinafter referred to as a “captured image”) to the drowsiness estimation device 1.

The drowsiness estimation device 1 includes a sensing unit 11, a first noise factor detecting unit 12, a feature amount calculating unit 13, a drowsiness score calculating unit 14, and a drowsiness estimation unit 15.

The feature amount calculating unit 13 includes a sensing result selecting unit 131.

The sensing unit 11 acquires information (hereinafter referred to as “drowsiness related information”) indicating a state related to drowsiness of the driver on the basis of a captured image obtained by imaging the face of the driver. Note that the sensing unit 11 acquires the captured image from the imaging device 2 in units of frames. The sensing unit 11 acquires the drowsiness related information for each frame.

In the first embodiment, processing of acquiring the drowsiness related information performed by the sensing unit 11 is referred to as “sensing processing”.

The state related to drowsiness of the driver includes how open the eyes of the driver are (in other words, an eyelid opening degree), how open the mouth of the driver is (in other words, an opening degree), positions of feature points of the face of the driver, a line-of-sight direction of the driver, a face direction of the driver, a head position of the driver, and the like. The feature points of the face are outer corners of the eyes, inner corners of the eyes, points on outer peripheries of the eyes, and the like, and the positions of the feature points of the driver's face are indicated by coordinates on the captured image, for example. The face direction of the driver is expressed by, for example, an angle on the basis of a case where the driver faces the front with respect to the traveling direction of the vehicle (0 degrees). The head position of the driver is represented by coordinates in a real space. Since the installation position and the angle of view of the imaging device 2 are known in advance, the sensing unit 11 can calculate the face direction and the head position of the driver from the captured image.

Note that, in the first embodiment, a state related to drowsiness of a driver is determined in advance by an administrator or the like.

It is sufficient that the sensing unit 11 detects a state related to drowsiness of the driver using a known image recognition technique and acquire the drowsiness related information.

The sensing unit 11 outputs the acquired drowsiness related information to the first noise factor detecting unit 12. At this time, for example, the sensing unit 11 may output the drowsiness related information to the first noise factor detecting unit 12 in association with a frame of the captured image that is the source of acquisition of the drowsiness related information. In the following description, the drowsiness related information or information in which the drowsiness related information and the frame of the captured image that is the acquisition source of the drowsiness related information are associated with each other, which is output from the sensing unit 11 to the first noise factor detecting unit 12, is also referred to as a “sensing result”. Note that it is assumed that information indicating imaging date and time is assigned to each frame of the captured image.

The first noise factor detecting unit 12 detects an action (hereinafter referred to as “noise factor action”) accompanied by eye movement of the driver, which is similar to an action caused by drowsiness on the basis of the drowsiness related information acquired by the sensing unit 11.

In the first embodiment, processing of detecting a noise factor performed by the first noise factor detecting unit 12 is referred to as “first noise factor detection processing”.

Examples of the noise factor action include, for example, an action of looking downward (what is called downward view) or an action of squinting.

For example, the driver may look down when looking at the meter.

In addition, for example, when sunlight is dazzling, the driver may have what is called “frowning face”. In the first embodiment, the what is called frowning face is also referred to as a “patient face”. A person generally squints their eyes when making a “patient face.” Also, for example, a person generally squints their eyes when they smile.

On the other hand, when drowsy, a person covers his/her eyes with eyelids to close his/her eyes, or closes his/her eyes to stop drowsiness. When the eye is covered with the eyelid or when the eye is closed, the eye becomes thinner.

As described above, it can be said that an action of what is called downward looking, an action of making a patient face, or an action of smiling is a noise factor action similar to the action of covering the eyes with the eyelids or closing the eyes caused by drowsiness.

The first noise factor detecting unit 12 detects such a noise factor action.

Note that what kind of action is set as the noise factor action is determined in advance by the administrator or the like.

For example, in a case where the noise factor action is an action of squinting eyes, the first noise factor detecting unit 12 detects that there is a noise factor action by the driver when the eyelid opening degree of the driver is equal to or less than a preset threshold (hereinafter referred to as “eyelid opening degree determination threshold”) on the basis of the drowsiness related information.

For example, the first noise factor detecting unit 12 may detect that there is a noise factor action by the driver when the eyelid opening degree of the driver becomes smaller than an eyelid opening degree based on the previous drowsiness related information by a preset threshold (hereinafter referred to as the “eyelid opening degree difference determination threshold”) or more on the basis of the drowsiness related information in time series. The first noise factor detecting unit 12 stores sensing results acquired from the sensing unit 11 in a storage unit (not illustrated) in time series. The first noise factor detecting unit 12 can specify the previous drowsiness related information on the basis of the sensing result stored in the storage unit. The storage unit is provided in a place that can be referred to by the drowsiness estimation device 1.

The eyelid opening degree determination threshold and the eyelid opening degree difference determination threshold are appropriately set by an administrator or the like and stored in the storage unit.

Note that, in this case, the drowsiness related information includes at least the eyelid opening degree of the driver.

Further, for example, the first noise factor detecting unit 12 may detect that there is a noise factor action by the driver using a model (hereinafter referred to as a “machine learning model”) in which the “patient face” has been learned in advance. The machine learning model used by the first noise factor detecting unit 12 to detect the noise factor action by the driver is also referred to as a “first machine learning model”. The first machine learning model is a machine learning model such as a support vector machine (SVM), a random forest, Light Gradient Boosting Machine (LightGBM), or a convolutional neural network.

The first machine learning model is a model that receives, as an input, information indicating a position of a feature point of the face of the person on the image, and outputs information indicating whether or not the person has a patient face. The first machine learning model is generated in advance and stored in the storage unit.

The first noise factor detecting unit 12 inputs the drowsiness related information to the first machine learning model and obtains information indicating whether or not the driver has a patient face, thereby detecting that the driver has a patient face.

Note that, in this case, the drowsiness related information includes at least the position information of the feature point of the driver's face on the captured image.

Further, for example, in a case where the noise factor action is action of looking down, the first noise factor detecting unit 12 detects that there is a noise factor action by the driver when the line-of-sight direction of the driver is equal to or less than a preset threshold (hereinafter referred to as a “line-of-sight direction determination threshold”) on the basis of the drowsiness related information.

For example, in a case where the line-of-sight direction of the driver changes downward by a preset angle (hereinafter, referred to as a “downward looking determination angle”) or more in a preset period (hereinafter, referred to as a “downward looking determination period”) on the basis of time-series drowsiness related information, the first noise factor detecting unit 12 may detect that there is a noise factor action by the driver.

The line-of-sight direction determination threshold, the downward looking determination period, and the downward looking determination angle are appropriately set in accordance with the installation position, the angle of view, and the like of the imaging device 2 by the administrator or the like and stored in the storage unit.

Note that, in this case, the drowsiness related information includes at least the line-of-sight direction of the driver.

When detecting that there is a noise factor action by the driver, the first noise factor detecting unit 12 outputs information (hereinafter referred to as “noise factor action information”) regarding the detected noise factor action to the feature amount calculating unit 13 together with the sensing result acquired from the sensing unit 11.

The noise factor action information includes information indicating that the first noise factor detecting unit 12 has detected the noise factor action, and drowsiness related information that is the source of detection of the noise factor action by the first noise factor detecting unit 12. The noise factor action information may further include information capable of specifying a type (looking downward, narrowing eyes, or the like) of the noise factor action detected by the first noise factor detecting unit 12.

The feature amount calculating unit 13 calculates a feature amount (hereinafter referred to as a “drowsiness estimation feature amount”) for estimating the drowsiness of the driver on the basis of the drowsiness related information (hereinafter referred to as “post-noise factor removal drowsiness related information”) after excluding the drowsiness related information that is the source of detection of the noise factor action by the first noise factor detecting unit 12 from the drowsiness related information acquired by the sensing unit 11.

In the first embodiment, processing of calculating the drowsiness estimation feature amount performed by feature amount calculating unit 13 is referred to as “feature amount calculation processing”.

The calculation of the drowsiness estimation feature amount by the feature amount calculating unit 13 will be described in detail.

First, on the basis of the noise factor action information related to the noise factor action detected by the first noise factor detecting unit 12, the sensing result selecting unit 131 included in the feature amount calculating unit 13 attaches an exclusion target flag to the drowsiness related information as an exclusion target in the calculation of the drowsiness estimation feature amount among the drowsiness related information acquired by the sensing unit 11.

For example, it is assumed that the first noise factor detecting unit 12 detects a noise factor action that is a squinting action. In this case, the sensing result selecting unit 131 sets, as the drowsiness related information as the exclusion target, the drowsiness related information that is the source of detection of the noise factor action by the first noise factor detecting unit 12 from among the drowsiness related information included in the sensing result. Then, the sensing result selecting unit 131 attaches the exclusion object flag to the drowsiness related information as the exclusion target in the sensing result.

As described above, the first noise factor detecting unit 12 outputs the sensing result acquired from the sensing unit 11 to the feature amount calculating unit 13 together with the noise factor action information. Therefore, the sensing result selecting unit 131 can specify the drowsiness related information as the exclusion target from the noise factor action information and the sensing result output from the first noise factor detecting unit 12.

For the sensing result output from the first noise factor detecting unit 12, if the drowsiness related information included in the sensing result is the drowsiness related information as the exclusion target, the sensing result selecting unit 131 attaches an exclusion target flag to the drowsiness related information. Then, the sensing result selecting unit 131 outputs, to the feature amount calculating unit 13, the sensing result after attaching the exclusion target flag to the drowsiness related information as the exclusion target.

Next, on the basis of the sensing result output from the sensing result selecting unit 131, the feature amount calculating unit 13 excludes the drowsiness related information as the exclusion target to which the exclusion target flag has been attached by the sensing result selecting unit 131 from the drowsiness related information acquired by the sensing unit 11, thereby specifying the drowsiness related information after excluding the drowsiness related information that is the source of detection of the noise factor action by the first noise factor detecting unit 12 from the drowsiness related information acquired by the sensing unit 11, that is, the post-noise factor removal drowsiness related information.

Then, the feature amount calculating unit 13 calculates a drowsiness estimation feature amount on the basis of the identified post-noise factor removal drowsiness related information.

That is, the feature amount calculating unit 13 calculates the drowsiness estimation feature amount on the basis of the drowsiness related information to which the exclusion target flag is not attached among the drowsiness related information acquired from the sensing unit 11 via the first noise factor detecting unit 12.

The drowsiness estimation feature amount includes, for example, an eyelid opening degree, a mouth opening degree, a face direction of a driver, a head position of the driver, a line-of-sight direction of the driver, and a PERCLOS (Percent of the time eyelids are closed, closed eye time rate per unit time) of the driver, the number of blinks of the driver, or the blink speed of the driver.

The feature amount calculating unit 13 calculates the drowsiness estimation feature amount as described above on the basis of the post-noise factor removal drowsiness related information.

Note that, in a case where the drowsiness estimation feature amount is a feature amount that needs to be determined from the background of the driver's past state, such as PERCLOS, the number of blinks, or the blinking speed, the feature amount calculating unit 13 stores, for example, the sensing result output from the sensing result selecting unit 131, and calculates the drowsiness estimation feature amount on the basis of a preset number of pieces of drowsiness related information in the past on the basis of the stored sensing result.

For example, the feature amount calculating unit 13 may calculate the drowsiness estimation feature amount on the basis of the stored post-noise factor removal drowsiness related information acquired in the past preset period (hereinafter referred to as a “feature amount calculation target period”).

For example, assuming that the feature amount calculation target period is three minutes, the feature amount calculating unit 13 calculates the drowsiness estimation feature amount on the basis of the post-noise factor removal drowsiness related information acquired in the past three minutes from the present.

In the post-noise factor removal drowsiness related information, the drowsiness related information as the exclusion target is excluded from the drowsiness related information acquired on the basis of the captured image acquired from the imaging device 2. Specifically, the drowsiness related information that is the source of detection of the noise factor action by the first noise factor detecting unit 12 is excluded from among the drowsiness related information acquired by the sensing unit 11 on the basis of the imaging device 2. That is, the feature amount calculating unit 13 does not use the drowsiness related information acquired on the basis of a frame of a captured image in which the driver taking the noise factor action is captured for the calculation of the drowsiness estimation feature amount. More specifically, the feature amount calculating unit 13 does not use the drowsiness related information acquired on the basis of the frame of the captured image in which the driver taking the noise factor action is captured for the calculation of the drowsiness estimation feature amount.

The feature amount calculating unit 13 outputs the drowsiness related information, more specifically, information (hereinafter referred to as “feature amount information”) in which the post-noise factor removal drowsiness related information is associated with the calculated drowsiness estimation feature amount to the drowsiness score calculating unit 14.

Note that, here, as described above, the sensing result selecting unit 131 gives the exclusion target flag to the drowsiness related information acquired by the sensing unit 11, and the feature amount calculating unit 13 specifies the post-noise factor removal drowsiness related information on the basis of the exclusion target flag, and calculates the drowsiness estimation feature amount from the specified post-noise factor removal drowsiness related information.

However, this is merely an example, and the feature amount calculating unit 13 may calculate the drowsiness estimation feature amount by another method.

For example, upon specifying the drowsiness related information as the exclusion target on the basis of the noise factor action information related to the noise factor action detected by the first noise factor detecting unit 12, the sensing result selecting unit 131 excludes the specified drowsiness related information as the exclusion target from among the drowsiness related information acquired by the sensing unit 11 included in the sensing result, and selects the drowsiness related information after exclusion as the post-noise factor removal drowsiness related information.

Upon selecting the post-noise factor removal drowsiness related information, the sensing result selecting unit 131 outputs the sensing result (hereinafter referred to as “post-noise removal sensing result”) including the selected post-noise factor removal drowsiness related information to the feature amount calculating unit 13.

The feature amount calculating unit 13 calculates a drowsiness estimation feature amount on the basis of the post-noise factor removal drowsiness related information output from the sensing result selecting unit 131.

That is, the feature amount calculating unit 13 calculates the drowsiness estimation feature amount on the basis of the drowsiness related information selected by the sensing result selecting unit 131 and included in the post-noise removal sensing result among the drowsiness related information acquired from the sensing unit 11 via the first noise factor detecting unit 12.

The feature amount calculating unit 13 may calculate the drowsiness estimation feature amount in this manner.

The drowsiness score calculating unit 14 calculates a drowsiness score using the drowsiness estimation feature amount calculated by the feature amount calculating unit 13. The drowsiness score calculating unit 14 can specify the drowsiness estimation feature amount calculated by the feature amount calculating unit 13 from the feature amount information output from the feature amount calculating unit 13.

The drowsiness score calculated by the drowsiness score calculating unit 14 is a score indicating the degree of drowsiness of the driver used for driver drowsiness estimation. In the first embodiment, as an example, the drowsiness score is represented by “0” to “100”, and the higher the drowsiness score, the higher the degree of drowsiness of the driver. Note that the drowsiness estimation unit 15 estimates the drowsiness of the driver using the drowsiness score.

In the first embodiment, processing of calculating the drowsiness score performed by the drowsiness score calculating unit 14 is referred to as “drowsiness score calculation processing”.

The drowsiness score calculating unit 14 calculates the drowsiness score using, for example, a machine learning model that has learned the drowsiness score in advance.

The machine learning model used by the drowsiness score calculating unit 14 to calculate the drowsiness score is also referred to as a “second machine learning model”. The second machine learning model is a machine learning model such as an SVM (support vector machine), a random forest, LightGBM (Light Gradient Boosting Machine), or a convolutional neural network.

For example, the second machine learning model receives the drowsiness estimation feature amount as an input, and outputs the drowsiness score. The second machine learning model is generated in advance and stored in a place that can be referred to by the drowsiness score calculating unit 14, such as a storage unit.

The drowsiness score calculating unit 14 calculates the drowsiness score by obtaining the drowsiness score by inputting the drowsiness estimation feature amount to the second machine learning model.

The drowsiness score calculating unit 14 may calculate the drowsiness score on the basis of, for example, a preset rule (hereinafter referred to as “drowsiness score calculation rule”) for calculating the drowsiness score.

A drowsiness score calculation rule is generated in advance by the administrator or the like, and is stored in a place that can be referred to by the drowsiness score calculating unit 14, such as a storage unit.

As the drowsiness score calculation rule, for example, a drowsiness score calculation rule in accordance with the number of blinks in the set period such as “the drowsiness score is set to “60” if the number of blinks in the past three minutes is equal to or more than 20 times” is set.

The drowsiness score calculating unit 14 outputs the calculated drowsiness score of the driver to the drowsiness estimation unit 15.

The drowsiness estimation unit 15 estimates the drowsiness of the driver on the basis of the drowsiness score calculated by the drowsiness score calculating unit 14.

The drowsiness of the driver estimated by the drowsiness estimation unit 15 may be expressed by a plurality of states such as “awakening”, “weak drowsiness”, and “strong drowsiness”, a binary value of “drowsiness is present” and “drowsiness is absent”, or a continuous value indicating a degree of drowsiness.

In the first embodiment, processing of estimating the drowsiness of the driver performed by the drowsiness estimation unit 15 is referred to as “drowsiness estimation processing”.

The drowsiness estimation unit 15 estimates the drowsiness of the driver using, for example, a machine learning model in which the drowsiness is already learned.

The machine learning model used by the drowsiness estimation unit 15 to estimate the drowsiness of the driver is also referred to as a “third machine learning model”. The third machine learning model is a machine learning model such as an SVM (support vector machine), a random forest, LightGBM (Light Gradient Boosting Machine), or a convolutional neural network.

The third machine learning model receives, for example, a drowsiness score as an input and outputs information indicating drowsiness. The information indicating drowsiness is, for example, information indicating a plurality of states of drowsiness of the driver, information indicating whether “drowsiness is present” or “drowsiness is absent”, or a continuous value indicating a degree of drowsiness.

The third machine learning model is generated in advance and stored in a place that can be referred to by the drowsiness estimation unit 15, such as a storage unit.

The drowsiness estimation unit 15 inputs the drowsiness score to the third machine learning model to obtain information indicating the drowsiness, thereby estimating the drowsiness of the driver.

The drowsiness estimation unit 15 may estimate the drowsiness of the driver on the basis of, for example, a preset rule (hereinafter referred to as “drowsiness estimation rule”) for estimating the drowsiness of the driver.

The drowsiness estimation rule is generated in advance by the administrator or the like, and stored in a place that can be referred to by the drowsiness estimation unit 15, such as a storage unit.

In the drowsiness estimation rule, for example, conditions are set in which the range of the drowsiness score is associated with the information indicating the plurality of states of the drowsiness of the driver such as “the drowsiness score of “0” to “50” is regarded as “awakening”, the drowsiness score of “50” to “60” is regarded as “weak drowsiness”, and the drowsiness score equal to or more than “60” is regarded as “strong drowsiness”, or a condition such as “the drowsiness score equal to or more than “60” is regarded as “drowsiness is present”, and the drowsiness score less than “60” is regarded as “drowsiness is absent”, or a calculation formula of the degree of drowsiness based on the drowsiness score is set.

The drowsiness estimation unit 15 outputs the estimation result (hereinafter referred to as “drowsiness estimation result”) of the drowsiness of the driver to an external device (not illustrated) of the drowsiness estimation device 1.

For example, the drowsiness estimation unit 15 outputs the drowsiness estimation result to an alarm device mounted on the vehicle. The alarm device outputs an alarm if the drowsy estimation result indicates that the driver is drowsy.

For example, the drowsiness estimation unit 15 may store the drowsiness estimation result in the storage unit.

An operation of the drowsiness estimation device 1 according to the first embodiment will be described.

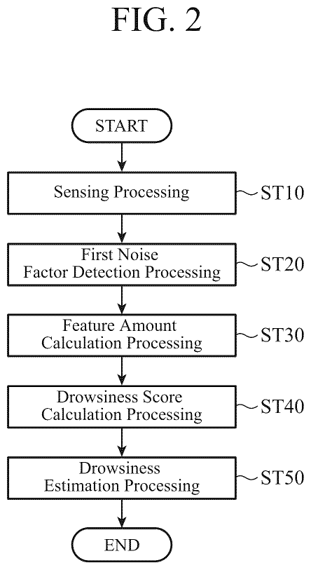

FIG. 2 is a flowchart for describing the operation of the drowsiness estimation device 1 according to the first embodiment.

For example, when the power of the vehicle is turned on and the captured image is output from the imaging device 2, the drowsiness estimation device 1 repeats the operation illustrated in the flowchart of FIG. 2 until the power of the vehicle is turned off.

The sensing unit 11 acquires a captured image obtained by imaging the face of the driver from the imaging device 2, and performs sensing processing of acquiring drowsiness related information on the basis of the acquired captured image (step ST10).

The sensing unit 11 outputs the sensing result to the first noise factor detecting unit 12.

The first noise factor detecting unit 12 performs first noise factor detection processing of detecting a noise factor action of the driver similar to an action caused by drowsiness on the basis of the drowsiness related information acquired by the sensing unit 11 in step ST10 (step ST20).

When detecting that there is a noise factor action by the driver, the first noise factor detecting unit 12 outputs the noise factor action information to the feature amount calculating unit 13 together with the sensing result acquired from the sensing unit 11.

Note that, when the first noise factor detecting unit 12 does not detect occurrence of the noise factor action by the driver, information indicating that occurrence of the noise factor action has not been detected may be output to the feature amount calculating unit 13, or nothing may be output to the feature amount calculating unit 13.

The feature amount calculating unit 13 performs feature amount calculation processing of calculating a drowsiness estimation feature amount on the basis of the post-noise factor removal drowsiness related information after excluding the drowsiness related information that is the source of detection of the noise factor action by the first noise factor detecting unit 12 in step ST20 from the drowsiness related information acquired by the sensing unit 11 in step ST10 (step ST30). More specifically, the feature amount calculating unit 13 performs the feature amount calculation processing of calculating the drowsiness estimation feature amount on the basis of the post-noise factor removal drowsiness related information after excluding the drowsiness related information that is the source of detection of the noise factor action by the first noise factor detecting unit 12 from the drowsiness related information acquired by the sensing unit 11.

The feature amount calculating unit 13 outputs the feature amount information to the drowsiness score calculating unit 14.

The drowsiness score calculating unit 14 performs drowsiness score calculation processing of calculating the drowsiness score using the drowsiness estimation feature amount calculated by the feature amount calculating unit 13 in step ST30 (step ST40).

The drowsiness score calculating unit 14 outputs the calculated drowsiness score of the driver to the drowsiness estimation unit 15.

The drowsiness estimation unit 15 performs drowsiness estimation processing of estimating the drowsiness of the driver on the basis of the drowsiness score calculated by the drowsiness score calculating unit 14 in step ST40 (step ST50).

The drowsiness estimation unit 15 outputs a drowsiness estimation result of the driver.

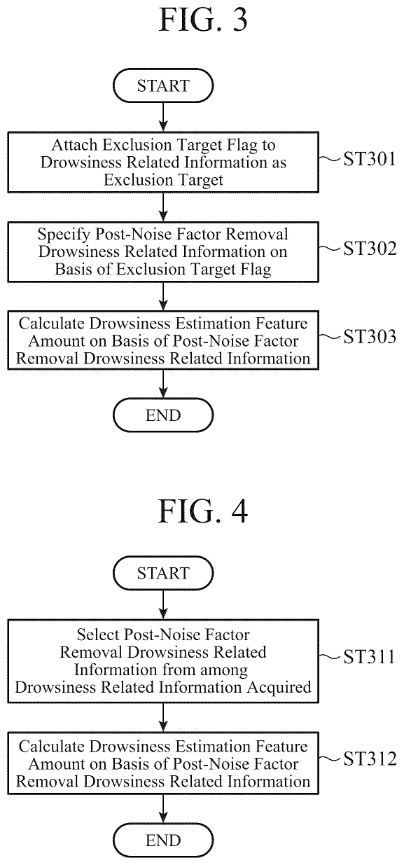

FIG. 3 is a flowchart for describing an example of detailed processing of step ST30 in FIG. 2.

More specifically, FIG. 3 is a flowchart for describing details of the feature amount calculation processing in a case where the feature amount calculating unit 13 specifies the post-noise factor removal drowsiness related information on the basis of the exclusion target flag attached to the drowsiness related information as the exclusion target by the sensing result selecting unit 131 and calculates a drowsiness estimation feature amount on the basis of the specified post-noise factor removal drowsiness related information in the first embodiment.

On the basis of the noise factor action information related to the noise factor action detected by the first noise factor detecting unit 12, the sensing result selecting unit 131 attaches the exclusion target flag to the drowsiness related information as the exclusion target in calculating the drowsiness estimation feature amount among the drowsiness related information acquired by the sensing unit 11 (step ST301).

The sensing result selecting unit 131 outputs, to the feature amount calculating unit 13, the sensing result after the exclusion target flag is assigned to the drowsiness related information as the exclusion target.

On the basis of the sensing result output from the sensing result selecting unit 131 in step ST301, the feature amount calculating unit 13 specifies the post-noise factor removal drowsiness related information by excluding the drowsiness related information as the exclusion target to which the exclusion target flag is attached by the sensing result selecting unit 131 from the drowsiness related information acquired by the sensing unit 11 (step ST302).

Then, the feature amount calculating unit 13 calculates the drowsiness estimation feature amount on the basis of the post-noise factor removal drowsiness related information specified in step ST302 (step ST303).

The feature amount calculating unit 13 outputs the feature amount information to the drowsiness score calculating unit 14.

FIG. 4 is a flowchart for describing another example of detailed processing of step ST30 in FIG. 2.

More specifically, FIG. 4 is a flowchart for describing details of feature amount calculation processing in a case where the feature amount calculating unit 13 calculates a drowsiness estimation feature amount on the basis of the post-noise factor removal drowsiness related information output from the sensing result selecting unit 131 in the first embodiment.

After specifying the drowsiness related information as the exclusion target on the basis of the noise factor action information related to the noise factor action detected by the first noise factor detecting unit 12, the sensing result selecting unit 131 excludes the specified drowsiness related information as the exclusion target from among the drowsiness related information acquired by the sensing unit 11 included in the sensing result, and selects the drowsiness related information after exclusion as the post-noise factor removal drowsiness related information (step ST311).

When selecting the post-noise factor removal drowsiness related information, the sensing result selecting unit 131 outputs post-noise removal sensing result to the feature amount calculating unit 13.

The feature amount calculating unit 13 calculates the drowsiness estimation feature amount on the basis of the post-noise factor removal drowsiness related information output from the sensing result selecting unit 131 in step ST311 (step ST312).

The feature amount calculating unit 13 outputs the feature amount information to the drowsiness score calculating unit 14.

As described above, the drowsiness estimation device 1 acquires the drowsiness related information for each frame on the basis of the frame of the captured image in which the face of the driver is captured, and detects a noise factor action similar to an action of the driver caused by drowsiness on the basis of the acquired drowsiness related information. The drowsiness estimation device 1 calculates the drowsiness estimation feature amount on the basis of the post-noise factor removal drowsiness related information after excluding the drowsiness related information that is the source of detection of the noise factor action from the acquired drowsiness related information. Then, the drowsiness estimation device 1 calculates the drowsiness score using the drowsiness estimation feature amount, and estimates the drowsiness of the driver on the basis of the calculated drowsiness score.

Actions of a person include an action having a feature similar to a feature when drowsiness occurs, such as what is called downward looking, what is called frowning, or smiling, in other words, a noise factor action.

In the related art, in estimating the drowsiness of the driver, if the driver takes a noise factor action as described above, the estimation accuracy of the drowsiness of the driver may decrease. More specifically, in the related art, when estimating the drowsiness of the driver, the feature amount calculated on the basis of the information of the face of the driver who takes the noise factor action, that is, the captured image in which the face of the driver is captured becomes noise, and there is a possibility that the estimation accuracy of the drowsiness of the driver decreases.

On the other hand, as described above, the drowsiness estimation device 1 calculates the drowsiness estimation feature amount on the basis of the post-noise factor removal drowsiness related information, and estimates the drowsiness of the driver on the basis of the drowsiness score calculated using the drowsiness estimation feature amount.

Therefore, in estimating the drowsiness of the driver, the drowsiness estimation device 1 can prevent a decrease in estimation accuracy of the drowsiness of the driver due to the driver taking a noise factor action. More specifically, when estimating drowsiness of the driver, the drowsiness estimation device 1 can prevent the estimation accuracy of the drowsiness of the driver from deteriorating due to use of a feature amount that becomes noise for estimation of the drowsiness of the driver, and can perform highly accurate drowsiness estimation.

Note that, in estimating the drowsiness of the driver, the feature amount that becomes noise is a feature amount calculated on the basis of the captured image obtained by capturing the face of the driver taking the noise factor action, and the feature amount calculated on the basis of the captured image obtained by capturing the face of the driver who is not taking the noise factor action is necessary for accurate driver drowsiness estimation.

Even if the driver takes a noise factor action such as downward looking, the drowsiness estimation device 1 removes only the feature amount that becomes noise calculated due to the noise factor action, and performs driver drowsiness estimation using the feature amount after removing the feature amount that becomes noise as the drowsiness estimation feature amount. That is, even if the driver takes the noise factor action, the drowsiness estimation device 1 does not stop the driver drowsiness estimation itself, but performs the drowsiness estimation using a necessary feature amount. Thus, when estimating drowsiness of the driver, the drowsiness estimation device 1 can prevent the estimation accuracy of the drowsiness of the driver from deteriorating due to use of a feature amount that becomes noise for estimation of the drowsiness of the driver, and can perform highly accurate drowsiness estimation.

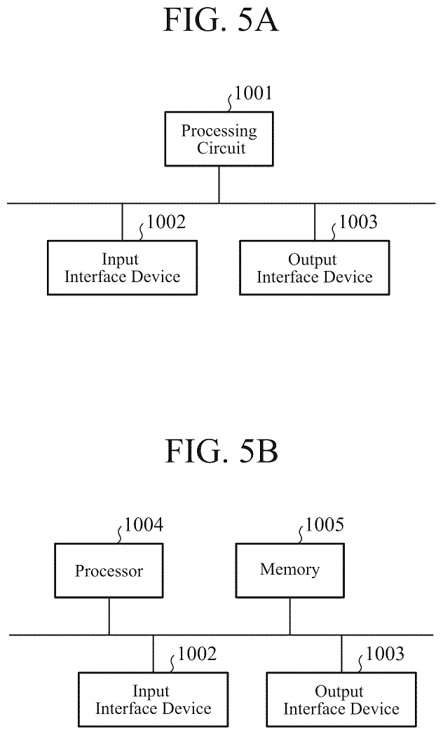

FIGS. 5A and 5B are diagrams illustrating an example of a hardware configuration of the drowsiness estimation device 1 according to the first embodiment.

In the first embodiment, the functions of the sensing unit 11, the first noise factor detecting unit 12, the feature amount calculating unit 13, the drowsiness score calculating unit 14, and the drowsiness estimation unit 15 are implemented by a processing circuit 1001. That is, the drowsiness estimation device 1 includes the processing circuit 1001 that performs control to estimate drowsiness of the driver using a feature amount excluding a feature amount calculated due to the driver taking the noise factor action as the drowsiness estimation feature amount on the basis of the captured image acquired from the imaging device 2.

The processing circuit 1001 may be dedicated hardware as illustrated in FIG. 5A or the processor 1004 that executes a program stored in a memory as illustrated in FIG. 5B.

In a case where the processing circuit 1001 is dedicated hardware, the processing circuit 1001 corresponds to, for example, a single circuit, a composite circuit, a programmed processor, a parallel programmed processor, an application specific integrated circuit (ASIC), a field-programmable gate array (FPGA), or a combination thereof.

When the processing circuit is the processor 1004, the functions of the sensing unit 11, the first noise factor detecting unit 12, the feature amount calculating unit 13, the drowsiness score calculating unit 14, and the drowsiness estimation unit 15 are implemented by software, firmware, or a combination of software and firmware. The software or firmware is described as a program and stored in a memory 1005. The processor 1004 reads and executes the program stored in the memory 1005 to execute the functions of the sensing unit 11, the first noise factor detecting unit 12, the feature amount calculating unit 13, the drowsiness score calculating unit 14, and the drowsiness estimation unit 15. That is, the drowsiness estimation device 1 includes the memory 1005 for storing a program that results in execution of steps ST10 to ST50 of FIG. 2 when executed by the processor 1004. In addition, it can also be said that the program stored in the memory 1005 causes a computer to execute a processing procedure or method of the sensing unit 11, the first noise factor detecting unit 12, the feature amount calculating unit 13, the drowsiness score calculating unit 14, and the drowsiness estimation unit 15. Here, the memory 1005 corresponds to, for example, a nonvolatile or volatile semiconductor memory such as a RAM, a read only memory (ROM), a flash memory, an erasable programmable read only memory (EPROM), or an electrically erasable programmable read-only memory (EEPROM), or a magnetic disk, a flexible disk, an optical disk, a compact disk, a mini disk, a digital versatile disc (DVD), or the like.

Note that a part of the functions of the sensing unit 11, the first noise factor detecting unit 12, the feature amount calculating unit 13, the drowsiness score calculating unit 14, and the drowsiness estimation unit 15 may be implemented by dedicated hardware, and a part thereof may be implemented by software or firmware. For example, the functions of the sensing unit 11 can be implemented by the processing circuit 1001 as dedicated hardware, and the functions of the first noise factor detecting unit 12, the feature amount calculating unit 13, the drowsiness score calculating unit 14, and the drowsiness estimation unit 15 can be implemented by the processor 1004 reading and executing the program stored in the memory 1005.

Furthermore, the drowsiness estimation device 1 includes an input interface device 1002 and an output interface device 1003 that perform wired communication or wireless communication with a device such as the imaging device 2.

In the first embodiment described above, the drowsiness estimation device 1 is an in-vehicle device mounted on a vehicle, and the sensing unit 11, the first noise factor detecting unit 12, the feature amount calculating unit 13, the drowsiness score calculating unit 14, and the drowsiness estimation unit 15 are included in the drowsiness estimation device 1.

The embodiment is not limited thereto, and a drowsiness estimation system may be configured by an in-vehicle device and a server in which a part of the sensing unit 11, the first noise factor detecting unit 12, the feature amount calculating unit 13, the drowsiness score calculating unit 14, and the drowsiness estimation unit 15 is mounted on the in-vehicle device of the vehicle and the others are provided in the server connected to the in-vehicle device via a network.

Alternatively, all of the sensing unit 11, the first noise factor detecting unit 12, the feature amount calculating unit 13, the drowsiness score calculating unit 14, and the drowsiness estimation unit 15 may be included in the server.

In addition, in the first embodiment described above, the subject is a driver of a vehicle as an example, but this is merely an example. The subject may be an occupant other than the driver of the vehicle. Furthermore, the subject may be an occupant including a driver of a mobile object other than a vehicle, such as a bus, a train, or an airplane. The drowsiness estimation device 1 according to the first embodiment can be applied as a drowsiness estimation device that estimates drowsiness of an occupant of a mobile object other than the vehicle.

As described above, according to the first embodiment, the drowsiness estimation device 1 includes the sensing unit 11 to acquire drowsiness related information indicating a state related to drowsiness of an occupant of a mobile object for each of frames on the basis of frames of a captured image obtained by capturing a face of the occupant, the first noise factor detecting unit 12 to detect a noise factor action of the occupant that is an action accompanied by eye movement and is similar to an action caused by drowsiness on the basis of the drowsiness related information acquired by the sensing unit 11, the feature amount calculating unit 13 to calculate a drowsiness estimation feature amount for estimating the drowsiness of the occupant on the basis of post-noise factor removal drowsiness related information after excluding the drowsiness related information that is a source of detection of the noise factor action by the first noise factor detecting unit 12 from the drowsiness related information acquired by the sensing unit 11, the drowsiness score calculating unit 14 to calculate a drowsiness score using the drowsiness estimation feature amount calculated by the feature amount calculating unit 13 and the drowsiness estimation unit 15 to estimate the drowsiness of the occupant on the basis of the drowsiness score calculated by the drowsiness score calculating unit 14. Therefore, in estimating drowsiness of the occupant of the mobile object, the drowsiness estimation device 1 can prevent a decrease in estimation accuracy of the drowsiness of the occupant due to that the occupant takes an action in which a feature similar to a feature when the drowsiness occurs is observed.

### Second Embodiment

Among traveling states of a mobile object, there is a traveling state in which it is assumed that drowsiness of an occupant hardly occurs.

If it is estimated that the occupant is drowsy in such a traveling state, this is erroneous estimation (over-estimation). If an alarm is issued on the basis of the erroneous estimation, the alarm is over-warning, and there is a possibility to cause annoyance to the occupant.

In a second embodiment, an embodiment will be described in which, in consideration of a traveling state of a mobile object, when it is estimated that drowsiness of an occupant is unlikely to occur from the traveling state of the mobile object, an estimation result of the drowsiness of the occupant is corrected in such a manner that the drowsiness of the occupant is not over-estimated.

Note that, in the following second embodiment, the subject is also assumed to be a driver of the vehicle.

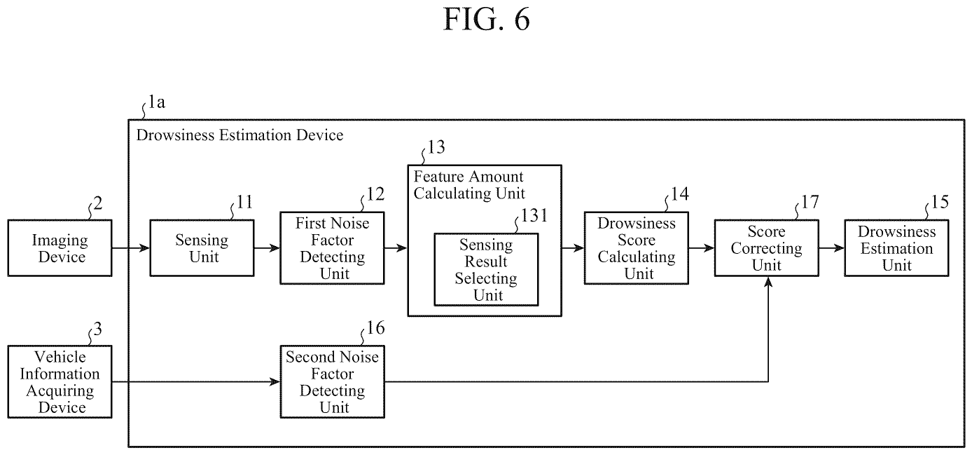

FIG. 6 is a diagram illustrating a configuration example of a drowsiness estimation device 1a according to the second embodiment.

The drowsiness estimation device 1a according to the second embodiment is connected to a vehicle information acquiring device 3 in addition to an imaging device 2.

The vehicle information acquiring device 3 outputs information related to the vehicle (hereinafter referred to as “vehicle information”) to the drowsiness estimation device 1a.

The vehicle information includes speed information of the vehicle, information indicating whether or not a blinker is used in the vehicle, in other words, whether or not the blinker is operated, information indicating a depression amount of a brake, information on a steering wheel angle, information indicating an accelerator opening degree, and the like.

The vehicle information acquiring device 3 includes, for example, a vehicle speed sensor that detects a speed of a vehicle, a blinker sensor that detects an operation state of a blinker, a brake sensor that detects a depression amount of a brake, a steering angle sensor that detects a steering wheel angle, and an accelerator sensor that detects an accelerator opening degree.

Note that, in FIG. 6, one vehicle information acquiring device 3 is connected to the drowsiness estimation device 1a, but this is merely an example, and a plurality of vehicle information acquiring devices 3 can be connected to the drowsiness estimation device 1a.

In the configuration of the drowsiness estimation device 1a according to the second embodiment, the same components as those of the drowsiness estimation device 1 described with reference to FIG. 1 in the first embodiment are denoted by the same reference numerals, and redundant description will be omitted.

The drowsiness estimation device 1a according to the second embodiment is different from the drowsiness estimation device 1 according to the first embodiment in that the drowsiness estimation device 1 includes a second noise factor detecting unit 16 and a score correcting unit 17.

The second noise factor detecting unit 16 acquires vehicle information from the vehicle information acquiring device 3, and detects a traveling state (hereinafter referred to as a “noise factor traveling state”) of the vehicle that is assumed to be unlikely to cause drowsiness in the driver on the basis of the acquired vehicle information.

In the second embodiment, processing of detecting the noise factor traveling state performed by the second noise factor detecting unit 16 is referred to as “second noise factor detection processing”.

Examples of the noise factor traveling state include a state in which the vehicle is traveling at a low speed, a state in which the blinker use frequency while the vehicle is traveling is high, a state in which the frequency of use of the brake in the vehicle is high, and a state in which a change in a steering wheel angle in the vehicle is large.

Note that which traveling state of the vehicle is set as the noise factor traveling state is determined in advance by the administrator or the like.

In a case where the vehicle is traveling at a low speed with a reduced speed, in a case where the blinker use frequency is high, for example, it is estimated that the vehicle is about to reach an intersection or is about to change lanes. In such a case, it is assumed that the driver hardly generates drowsiness.

Further, in a case where the frequency of use of the brake in the vehicle is high, for example, it is estimated that the vehicle is in a traffic jam.

Furthermore, in a case where there are many changes in the steering wheel angle in the vehicle, for example, it is estimated that the vehicle is traveling in a parking lot or an intersection.

In the traveling state of the vehicle as described above, it is assumed that the driver hardly generates drowsiness.

The administrator or the like obtains such information in advance by performing an experiment or the like.

Then, the administrator or the like determines the traveling state of the vehicle as described above, in which it is assumed that the driver hardly generates drowsiness, as the noise factor traveling state, and stores information regarding the noise factor traveling state in a place where the noise factor traveling state can be referred to, such as a storage unit.

The information regarding the noise factor traveling state is information for the second noise factor detecting unit 16 to determine the noise factor traveling state, and for example, a condition of the vehicle information indicating that the vehicle is in the noise factor traveling state is set. The condition of the vehicle information indicating that the vehicle is in the noise factor traveling state is, for example, that the vehicle speed is equal to or less than a preset threshold, that the blinker use frequency in a preset period is equal to or more than a preset threshold, that the brake use frequency in the preset period is equal to or more than a preset threshold, and that the change amount of the steering wheel angle in the preset period is equal to or more than a preset threshold.

Note that the second noise factor detecting unit 16 stores the vehicle information acquired from the vehicle information acquiring device 3 in a storage unit or the like in time series. The second noise factor detecting unit 16 can determine a change in the traveling state of the vehicle such as a change in the steering wheel angle on the basis of the stored vehicle information.

When detecting that the traveling state of the vehicle is the noise factor traveling state, the second noise factor detecting unit 16 outputs information (hereinafter referred to as “noise factor traveling state information”) on the detected noise factor traveling state to the score correcting unit 17 together with the vehicle information acquired from the vehicle information acquiring device 3.

The noise factor traveling state information includes information indicating that the second noise factor detecting unit 16 has detected the noise factor traveling state. The noise factor traveling state information may further include information capable of specifying the type (low-speed traveling, the blinker use frequency is high, the brake use frequency is high, the steering wheel angle is large, or the like) of the noise factor traveling state detected by the second noise factor detecting unit 16.

When the second noise factor detecting unit 16 detects the noise factor traveling state, the score correcting unit 17 corrects the drowsiness score calculated by the drowsiness score calculating unit 14.

Note that, in the second embodiment, the drowsiness score calculating unit 14 outputs the calculated drowsiness score of the driver to the score correcting unit 17.

The score correcting unit 17 corrects the drowsiness score according to a predetermined rule (hereinafter referred to as a “score correction rule”).

The score correction rule is set in advance by the administrator or the like, and is stored in a place that can be referred to by the score correcting unit 17, such as a storage unit.

In the second embodiment, processing of correcting the drowsiness score performed by the score correcting unit 17 is referred to as “drowsiness score correction processing”.

A specific example of correction of the drowsiness score by the score correcting unit 17 in the second embodiment will be described.

For example, it is assumed that a rule “drowsiness score is multiplied by 0.5 when noise factor traveling state is detected” is set in the score correction rule.

For example, it is assumed that the drowsiness score calculated by the drowsiness score calculating unit 14 is “90”. For example, it is assumed that the second noise factor detecting unit 16 now detects a noise factor traveling state in which the blinker use frequency is high because the blinker use frequency in the preset period is equal to or higher than the preset threshold.

Note that, when the vehicle is in a traveling state where the blinker use frequency is high, for example, it is conceivable that the vehicle frequently changes lanes. When such a traveling state of the vehicle is detected as the noise factor traveling state, it is assumed that the driver is in a state in which it is difficult to generate drowsiness. Nevertheless, the fact that the drowsiness score calculating unit 14 calculates the drowsiness score as high as “90” indicates that, for example, by some behavior of the driver such as the driver checking the left and right when frequently performing a lane change and this being regarded as an eye closing action (that is, an action when drowsiness occurs), it is conceivable that the feature amount calculating unit 13 may have calculated a feature amount with which the drowsiness score can be calculated high. That is, there is a possibility that the drowsiness score calculated by the drowsiness score calculating unit 14 is an error.

In this case, the score correcting unit 17 corrects the drowsiness score to “45”.

For example, it is assumed that it is determined that the drowsiness estimation unit 15 estimates that “drowsiness is present” when the drowsiness score exceeds “50”. In this case, when the drowsiness score is “90”, the drowsiness estimation unit 15 estimates that “drowsiness is present”. Then, for example, an alarm device, which is not illustrated, outputs an alarm on the basis of a drowsiness estimation result that “drowsiness is present” by the drowsiness estimation unit 15.

In the above-described example, in a case where the score correcting unit 17 does not correct the drowsiness score, an alarm against drowsiness is output to the driver although it is assumed that the driver is not drowsy.

The score correcting unit 17 corrects the drowsiness score when the second noise factor detecting unit 16 detects the noise factor traveling state, so that the drowsiness estimation unit 15 can prevent over-estimation of the drowsiness of the driver and suppress the over-warning.

Note that the above-described specific example is merely an example.

In the score correction rule, a rule different from the content described in the above example may be set.

For example, the score correction rule may be set in such a manner that the degree of correcting the drowsiness score varies in accordance with the type of the noise factor traveling state.

For example, a rule “the drowsiness score is multiplied by 0.5 when a noise factor traveling state in which the blinker is frequently used is detected, and the drowsiness score is multiplied by 0.7 when a noise factor traveling state that is a low-speed traveling state is detected” may be set in the score correction rule.

The score correcting unit 17 outputs the drowsiness score after correction (hereinafter referred to as “post-correction drowsiness score”) to the drowsiness estimation unit 15.

The drowsiness estimation unit 15 estimates the drowsiness of the driver on the basis of the post-correction drowsiness score.

An operation of the drowsiness estimation device 1a according to the second embodiment will be described.

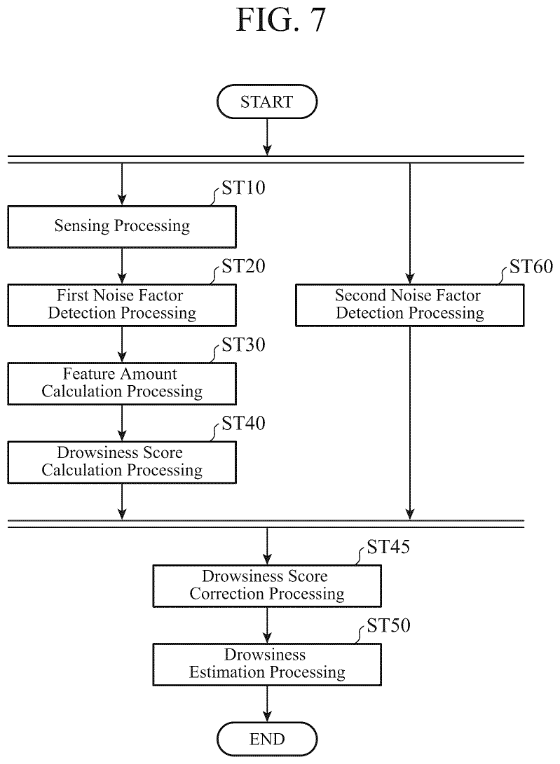

FIG. 7 is a flowchart for describing the operation of the drowsiness estimation device 1a according to the second embodiment.

For example, when the power of the vehicle is turned on and the captured image is output from the imaging device 2, the drowsiness estimation device 1a repeats the operation illustrated in the flowchart of FIG. 7 until the power of the vehicle is turned off.

Specific operations in the processing of steps ST10 to ST40 and step ST50 in FIG. 7 are similar to the specific operations in the processing of steps ST10 to ST40 and step ST50 in FIG. 2 described in the first embodiment, respectively, and thus the same step numbers are given and redundant description is omitted.

The second noise factor detecting unit 16 acquires vehicle information from the vehicle information acquiring device 3, and performs the second noise factor detection processing of detecting a noise factor traveling state on the basis of the acquired vehicle information (step ST60).

When detecting that the traveling state of the vehicle is the noise factor traveling state, the second noise factor detecting unit 16 outputs the noise factor traveling state information to the score correcting unit 17 together with the vehicle information acquired from the vehicle information acquiring device 3.

Note that, when the second noise factor detecting unit 16 has not detected that the traveling state of the vehicle is the noise factor traveling state, information indicating that the noise factor traveling state has not been detected may be output to the score correcting unit 17, or nothing may be output to the score correcting unit 17.

When the second noise factor detecting unit 16 detects the noise factor traveling state in step ST60, the score correcting unit 17 performs drowsiness score correction processing of correcting the drowsiness score calculated by the drowsiness score calculating unit 14 (step ST45).

The score correcting unit 17 outputs the post-correction drowsiness score to the drowsiness estimation unit 15.

Note that, in the flowchart of FIG. 7, the processing of steps ST10 to ST40 and the processing of step ST60 are performed in parallel, but this is merely an example.

For example, the drowsiness estimation device 1a may execute the processing of step ST60 after the processing of steps ST10 to ST40.

It is sufficient that the processing of steps ST10 to ST40 and ST60 is performed before the processing of step ST45 is performed.

As described above, the drowsiness estimation device 1a acquires the drowsiness related information for each frame on the basis of the frame of the captured image in which the face of the driver is captured, and detects a noise factor action similar to an action of the driver caused by drowsiness on the basis of the acquired drowsiness related information. The drowsiness estimation device 1a calculates the drowsiness estimation feature amount on the basis of the post-noise factor removal drowsiness related information after excluding the drowsiness related information from which the noise factor action has been detected from the acquired drowsiness related information. Then, drowsiness estimation device 1a calculates the drowsiness score using the drowsiness estimation feature amount, and estimates the drowsiness of the driver on the basis of the calculated drowsiness score.

Furthermore, the drowsiness estimation device 1a corrects the calculated drowsiness score when detecting the noise factor traveling state on the basis of the vehicle information. When the drowsiness score is corrected, the drowsiness estimation device 1a estimates the drowsiness of the driver on the basis of the post-correction drowsiness score.

Therefore, in estimating the drowsiness of the driver, the drowsiness estimation device 1a can prevent the estimation accuracy of the drowsiness of the driver from deteriorating due to the driver taking the noise factor action, and more specifically, in estimating the drowsiness of the driver, the drowsiness estimation device 1a can prevent the estimation accuracy of the drowsiness of the driver from deteriorating due to use of a feature amount that becomes noise for estimation of the drowsiness of the driver, perform accurate drowsiness estimation, and prevent over-estimation of the drowsiness of the driver.

The hardware configuration of the drowsiness estimation device 1a according to the second embodiment is similar to the hardware configuration of the drowsiness estimation device 1 described with reference to FIGS. 5A and 5B in the first embodiment, and thus is not illustrated.

In the second embodiment, the functions of the sensing unit 11, the first noise factor detecting unit 12, the feature amount calculating unit 13, the drowsiness score calculating unit 14, the drowsiness estimation unit 15, the second noise factor detecting unit 16, and the score correcting unit 17 are implemented by the processing circuit 1001. That is, the drowsiness estimation device 1a includes the processing circuit 1001 that performs control to estimate the drowsiness of the driver by using, as the drowsiness estimation feature amount, a feature amount excluding a feature amount calculated due to the driver taking the noise factor action on the basis of the captured image acquired from the imaging device 2, and performs control to correct the drowsiness score on the basis of the vehicle information acquired from the vehicle information acquiring device 3.

The processing circuit 1001 reads and executes the program stored in the memory 1005 to execute the functions of the sensing unit 11, the first noise factor detecting unit 12, the feature amount calculating unit 13, the drowsiness score calculating unit 14, the drowsiness estimation unit 15, the second noise factor detecting unit 16, and the score correcting unit 17. That is, the drowsiness estimation device 1a includes the memory 1005 for storing the program that results in execution of steps ST10 to ST60 of FIG. 7 described above when executed by the processing circuit 1001. It can also be said that the program stored in the memory 1005 causes a computer to execute a procedure or a method of the sensing unit 11, the first noise factor detecting unit 12, the feature amount calculating unit 13, the drowsiness score calculating unit 14, the drowsiness estimation unit 15, the second noise factor detecting unit 16, and the score correcting unit 17.

The drowsiness estimation device 1a includes an input interface device 1002 and an output interface device 1003 that perform wired communication or wireless communication with devices such as the imaging device 2 and the vehicle information acquiring device 3.

In the second embodiment described above, the drowsiness estimation device 1a is an in-vehicle device mounted on a vehicle, and the sensing unit 11, the first noise factor detecting unit 12, the feature amount calculating unit 13, the drowsiness score calculating unit 14, the drowsiness estimation unit 15, the second noise factor detecting unit 16, and the score correcting unit 17 are included in the drowsiness estimation device 1a.

The embodiment is not limited thereto, and a drowsiness estimation system may be configured by an in-vehicle device and a server in which a part of the sensing unit 11, the first noise factor detecting unit 12, the feature amount calculating unit 13, the drowsiness score calculating unit 14, the drowsiness estimation unit 15, the second noise factor detecting unit 16, and the score correcting unit 17 is mounted on the in-vehicle device of the vehicle and the others are provided in the server connected to the in-vehicle device via a network.

In addition, all of the sensing unit 11, the first noise factor detecting unit 12, the feature amount calculating unit 13, the drowsiness score calculating unit 14, the drowsiness estimation unit 15, the second noise factor detecting unit 16, and the score correcting unit 17 may be included in the server.

Further, in the second embodiment described above, the subject is a driver of the vehicle as an example, but this is merely an example. The subject may be an occupant other than the driver of the vehicle. Furthermore, the subject may be an occupant including a driver of a mobile object other than a vehicle, such as a bus, a train, or an airplane. The drowsiness estimation device 1a according to the second embodiment can be applied as a drowsiness estimation device that estimates drowsiness of an occupant of a mobile object other than the vehicle.

As described above, according to the second embodiment, the drowsiness estimation device 1a includes the sensing unit 11 to acquire drowsiness related information indicating a state related to drowsiness of an occupant of a mobile object for each of frames on the basis of frames of a captured image obtained by capturing a face of the occupant, the first noise factor detecting unit 12 to detect a noise factor action that is an action accompanied by eye movement of the occupant and is similar to an action caused by drowsiness on the basis of the drowsiness related information acquired by the sensing unit 11, the feature amount calculating unit 13 to calculate a drowsiness estimation feature amount for estimating the drowsiness of the occupant on the basis of post-noise factor removal drowsiness related information after excluding the drowsiness related information that is a source of detection of the noise factor action by the first noise factor detecting unit 12 from the drowsiness related information acquired by the sensing unit 11, the drowsiness score calculating unit 14 to calculate a drowsiness score using the drowsiness estimation feature amount calculated by the feature amount calculating unit 13 and the drowsiness estimation unit 15 to estimate the drowsiness of the occupant on the basis of the drowsiness score calculated by the drowsiness score calculating unit 14. Therefore, in estimating drowsiness of the occupant of the mobile object, the drowsiness estimation device 1a can prevent a decrease in estimation accuracy of the drowsiness of the occupant due to that the occupant takes an action in which a feature similar to a feature when the drowsiness occurs is observed.

Furthermore, the drowsiness estimation device 1a includes the second noise factor detecting unit 16 to detect a noise factor traveling state on the basis of mobile object information related to the mobile object, the noise factor traveling state being a traveling state of the mobile object that is assumed to be unlikely to cause the drowsiness in the occupant, and the score correcting unit 17 to correct the drowsiness score calculated by the drowsiness score calculating unit 14 when the second noise factor detecting unit 16 detects the noise factor traveling state, in which the drowsiness estimation unit 15 estimates the drowsiness of the occupant on the basis of a post-correction drowsiness score after correction by the score correcting unit 17 when the score correcting unit 17 corrects the drowsiness score calculated by the drowsiness score calculating unit 14. Therefore, the drowsiness estimation device 1a can prevent over-estimation of drowsiness of the occupant.

### Third Embodiment

In the second embodiment, the drowsiness estimation device detects a noise factor traveling state on the basis of vehicle information, and corrects a drowsiness score when detecting the noise factor traveling state, thereby preventing erroneous estimation (over-estimation) of drowsiness.

On the other hand, malfunction of the sensing unit can also be considered as an event that may cause erroneous estimation of drowsiness. For example, in a situation where the driver of the vehicle is driving with eyes open while the behavior of the eyes is normal, there is a possibility that it is erroneously detected that the eyes are closed due to a mistake in the sensing processing by the sensing unit, and erroneous drowsiness related information is acquired. If the feature amount calculating unit calculates the drowsiness estimation feature amount on the basis of the erroneous drowsiness related information, for example, the drowsiness estimation feature amount that can estimate that “drowsiness is present” can be calculated. As a result, the drowsiness estimation unit may over-estimate the drowsiness of the driver.

In a third embodiment, an embodiment in which the drowsiness score is corrected in consideration of the possibility of such a mistake in sensing processing by the sensing unit will be described.

Note that, in the third embodiment described below, the subject is assumed to be a driver of the vehicle.

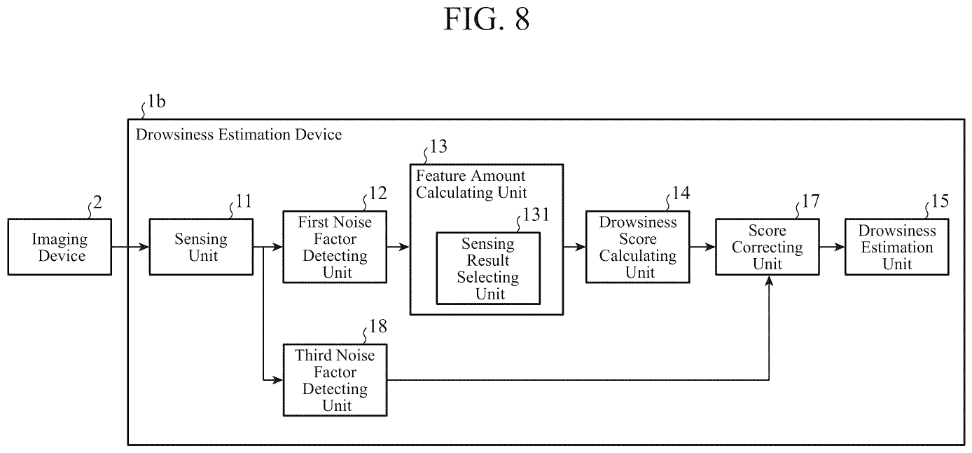

FIG. 8 is a diagram illustrating a configuration example of a drowsiness estimation device 1b according to the third embodiment.

In the configuration of the drowsiness estimation device 1b according to the third embodiment, the same components as those of the drowsiness estimation device 1 described with reference to FIG. 1 in the first embodiment are denoted by the same reference numerals, and redundant description will be omitted.

The drowsiness estimation device 1b according to the third embodiment is different from the drowsiness estimation device 1 according to the first embodiment in that the drowsiness estimation device 1b includes a third noise factor detecting unit 18 and a score correcting unit 17.

The third noise factor detecting unit 18 detects, on the basis of the drowsiness related information acquired by the sensing unit 11, occurrence of an event (hereinafter referred to as “noise factor sensing”) in which drowsiness related information is estimated to be erroneously acquired because the sensing unit 11 erroneously detects the state related to the drowsiness of the occupant.

Note that, in the third embodiment, the sensing unit 11 outputs the sensing result to the first noise factor detecting unit 12 and the third noise factor detecting unit 18.

In the third embodiment, processing of detecting the noise factor sensing performed by the third noise factor detecting unit 18 is referred to as “third noise factor detection processing”.

As an example of the noise factor sensing, there is an erroneous acquisition of drowsiness related information due to erroneous detection that the sensing unit 11 erroneously detects that, for example, the driver closes the eyes, in other words, the eyelid opening degree is low even though the eyes are actually open. Such erroneous acquisition of the drowsiness related information may lead to erroneous estimation of the drowsiness of the driver when the drowsiness estimation unit 15 estimates the drowsiness of the driver on the basis of the drowsiness related information.

The third noise factor detecting unit 18 detects whether or not the noise factor sensing is occurring, for example, on the basis of whether or not the drowsiness related information that can be determined as extremely long continuous eye closing is acquired on the basis of time-series drowsiness related information acquired by the sensing unit 11. The third noise factor detecting unit 18 detects the occurrence of the noise factor sensing when the drowsiness related information that can be determined as extremely long continuous eye closing is acquired.

For example, if a state in which the eyes are determined to be closed, more specifically, a state in which the eyelid opening degree is equal to or less than a preset threshold (hereinafter referred to as “eyelid opening degree for continuous eye closing determination”) continues for a preset period (hereinafter referred to as a “continuous eye closing determination period”) on the basis of the time-series drowsiness related information, the third noise factor detecting unit 18 detects that the drowsiness related information that can be regarded as extremely long continuous eye closing has been acquired, that is, the noise factor sensing has occurred. Note that the eyelid opening degree for continuous eye closing determination and the continuous eye closing determination period are determined in advance by the administrator or the like, and are stored in a place that can be referred to by the third noise factor detecting unit 18. Note that the third noise factor detecting unit 18 is only required to acquire the time-series drowsiness related information from sensing results stored in the storage unit.

As another example of the noise factor sensing, for example, the sensing unit 11 repeatedly erroneously detects that the driver closes the eyes even though the eyes are actually opened and erroneously detects that the driver opens the eyes even though the eyes are actually closed, thereby erroneously acquiring the drowsiness related information. Such erroneous acquisition of the drowsiness related information may lead to erroneous estimation of the drowsiness of the driver when the drowsiness estimation unit 15 estimates the drowsiness of the driver on the basis of the drowsiness related information.

For example, the third noise factor detecting unit 18 detects whether or not the noise factor sensing is occurring on the basis of whether or not the drowsiness related information that can be determined to have an extremely large number of blinks has been acquired on the basis of the time-series drowsiness related information acquired by the sensing unit 11. The third noise factor detecting unit 18 detects the occurrence of the noise factor sensing when the drowsiness related information that can be determined to have an extremely large number of blinks has been acquired.

For example, the third noise factor detecting unit 18 detects the blink of the driver by a known blink detection method on the basis of the time-series drowsiness related information. When blinks of a preset number of times (hereinafter referred to as a “blink frequency determination threshold”) or more are detected in a preset period (hereinafter referred to as a “blink determination period”), the third noise factor detecting unit 18 detects that the drowsiness related information that can be determined to have an extremely large number of blinks has been acquired, that is, the noise factor sensing has occurred. Note that the blink determination period and the blink frequency determination threshold are determined in advance by the administrator or the like and stored in a place that can be referred to by the third noise factor detecting unit 18.

Note that the above-described example is merely an example, and the condition for the third noise factor detecting unit 18 to detect that the noise factor sensing has occurred is appropriately determined in advance by the administrator or the like.

When detecting that the noise factor sensing has occurred, the third noise factor detecting unit 18 outputs information (hereinafter referred to as “noise factor sensing information”) regarding the detected noise factor sensing to the score correcting unit 17 together with the sensing result.

The noise factor sensing information includes information indicating that the third noise factor detecting unit 18 has detected that the noise factor sensing has occurred. The noise factor sensing information may further include information capable of specifying the type (extremely continuous eye closing, extreme blinking, or the like) of the noise factor sensing detected by the third noise factor detecting unit 18 as being generated.

When the third noise factor detecting unit 18 detects the noise factor sensing, the score correcting unit 17 corrects the drowsiness score calculated by the drowsiness score calculating unit 14 on the basis of the noise factor sensing.

Note that, in the third embodiment, the drowsiness score calculating unit 14 outputs the calculated drowsiness score of the driver to the score correcting unit 17.

The score correcting unit 17 corrects the drowsiness score according to a predetermined score correction rule.

In the third embodiment, processing of correcting the drowsiness score performed by the score correcting unit 17 is referred to as “drowsiness score correction processing”.

A specific example of correction of the drowsiness score by the score correcting unit 17 in the third embodiment will be described.

For example, it is assumed that a rule “drowsiness score is multiplied by 0.5 when occurrence of the noise factor sensing is detected” is set in the score correction rule.

For example, it is assumed that the drowsiness score calculated by the drowsiness score calculating unit 14 is “90”. In addition, for example, it is assumed that the third noise factor detecting unit 18 now detects that the noise factor sensing, which is acquisition of the drowsiness related information that can be regarded as an extremely long continuous eye closing, has occurred since a state in which the eyelid opening degree is equal to or less than the eyelid opening degree for continuous eye closing determination has continued for the continuous eye closing determination period (for example, 10 seconds) on the basis of the drowsiness related information acquired from the sensing unit 11.

It is abnormal to close eyes continuously for 10 seconds, but it is considered that the fact that the drowsiness score calculating unit 14 has calculated the high drowsiness score of “90” despite the abnormal state means that the continuous eye closing state, which is the abnormal state, has been regarded as a state in which drowsiness occurs, and the feature amount calculating unit 13 has calculated the feature amount with which the drowsiness score can be calculated to be high. That is, there is a possibility that the drowsiness score calculated by the drowsiness score calculating unit 14 is an error.

In this case, the score correcting unit 17 corrects the drowsiness score to “45”.

For example, it is assumed that the drowsiness estimation unit 15 estimates that “drowsiness is present” when the drowsiness score exceeds “50”. In this case, when the drowsiness score is “90”, the drowsiness estimation unit 15 estimates that “drowsiness is present”. Then, for example, an alarm device, which is not illustrated, outputs an alarm on the basis of a drowsiness estimation result that “drowsiness is present” by the drowsiness estimation unit 15.

In the above-described example, in a case where the score correcting unit 17 does not correct the drowsiness score, an alarm against drowsiness is output to the driver although it is assumed that the driver is not drowsy.

The score correcting unit 17 corrects the drowsiness score when the third noise factor detecting unit 18 detects the noise factor sensing, so that the drowsiness estimation unit 15 can prevent over-estimation of the drowsiness of the driver and suppress the over-warning.

Note that the above-described specific example is merely an example.

In the score correction rule, a rule different from the content described in the above example may be set.

Furthermore, for example, the score correction rule may be set in such a manner that the degree of correcting the drowsiness score varies in accordance with the type of noise factor sensing.

For example, a rule “drowsiness score is multiplied by 0.5 when occurrence of noise factor sensing that is erroneous acquisition of the drowsiness related information that can be regarded as an extremely long continuous eye closing is detected, and drowsiness score is multiplied by 0.7 when noise factor sensing that is erroneous acquisition of drowsiness related information that can be regarded as an extremely large number of blinks is detected” may be set in the score correction rule.

The score correcting unit 17 outputs the post-correction drowsiness score to the drowsiness estimation unit 15.

The drowsiness estimation unit 15 estimates the drowsiness of the driver on the basis of the post-correction drowsiness score.

An operation of the drowsiness estimation device 1b according to the third embodiment will be described.

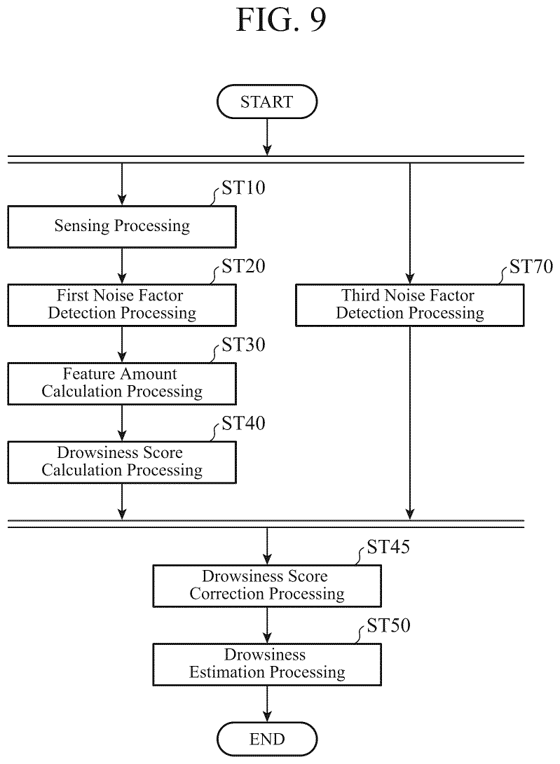

FIG. 9 is a flowchart for describing the operation of the drowsiness estimation device 1b according to the third embodiment.

For example, when the power of the vehicle is turned on and the captured image is output from the imaging device 2, the drowsiness estimation device 1b repeats the operation illustrated in the flowchart of FIG. 9 until the power of the vehicle is turned off.

Specific operations in the processing of steps ST10 to ST40 and step ST50 in FIG. 9 are similar to the specific operations in the processing of steps ST10 to ST40 and step ST50 in FIG. 2 described in the first embodiment, respectively, and thus the same step numbers are given and redundant description is omitted.

The third noise factor detecting unit 18 acquires the drowsiness related information from the sensing unit 11, and performs third noise factor detection processing of detecting the occurrence of the noise factor sensing on the basis of the acquired drowsiness related information (step ST70).

When detecting the occurrence of the noise factor sensing, the third noise factor detecting unit 18 outputs the noise factor sensing information from the sensing unit 11 to the score correcting unit 17 together with the drowsiness related information.

Note that, when the third noise factor detecting unit 18 has not detected the occurrence of the noise factor sensing, information indicating that occurrence of the noise factor sensing has not been detected may be output to the score correcting unit 17, or nothing may be output to the score correcting unit 17.

When the third noise factor detecting unit 18 detects the noise factor sensing in step ST70, the score correcting unit 17 performs drowsiness score correction processing of correcting the drowsiness score calculated by the drowsiness score calculating unit 14 on the basis of the noise factor sensing (step ST45).

The score correcting unit 17 outputs the post-correction drowsiness score to the drowsiness estimation unit 15.

Note that, in the flowchart of FIG. 9, the processing of steps ST10 to ST40 and the processing of step ST70 are performed in parallel, but this is merely an example.

For example, the drowsiness estimation device 1b may execute the processing of step ST70 after the processing of steps ST10 to ST40.

It is sufficient that the processing of steps ST10 to ST40 and ST70 is performed before the processing of step ST45 is performed.

As described above, the drowsiness estimation device 1b acquires the drowsiness related information for each frame on the basis of the frame of the captured image in which the face of the driver is captured, and detects a noise factor action similar to an action of the driver caused by drowsiness on the basis of the acquired drowsiness related information. The drowsiness estimation device 1b calculates the drowsiness estimation feature amount on the basis of the post-noise factor removal drowsiness related information after excluding the drowsiness related information from which the noise factor action has been detected from the acquired drowsiness related information. Then, the drowsiness estimation device 1b calculates the drowsiness score using the drowsiness estimation feature amount, and estimates the drowsiness of the driver on the basis of the calculated drowsiness score.

Furthermore, the drowsiness estimation device 1b corrects the drowsiness score when detecting the occurrence of the noise factor sensing on the basis of the drowsiness related information. When the drowsiness score is corrected, the drowsiness estimation device 1b estimates the drowsiness of the driver on the basis of the post-correction drowsiness score.

Therefore, in estimating the drowsiness of the driver, the drowsiness estimation device 1b can prevent the estimation accuracy of the drowsiness of the driver from deteriorating due to the driver taking the noise factor action, and more specifically, in estimating the drowsiness of the driver, the drowsiness estimation device 1b can prevent the estimation accuracy of the drowsiness of the driver from deteriorating due to use of a feature amount that becomes noise for estimation of the drowsiness of the driver, perform accurate drowsiness estimation, and prevent over-estimation of the drowsiness of the driver.

The hardware configuration of the drowsiness estimation device 1b according to the third embodiment is similar to the hardware configuration of the drowsiness estimation device 1 described with reference to FIGS. 5A and 5B in the first embodiment, and thus is not illustrated.

In the third embodiment, the functions of the sensing unit 11, the first noise factor detecting unit 12, the feature amount calculating unit 13, the drowsiness score calculating unit 14, the drowsiness estimation unit 15, the score correcting unit 17, and the third noise factor detecting unit 18 are implemented by the processing circuit 1001. That is, the drowsiness estimation device 1b includes the processing circuit 1001 for performing control to estimate the drowsiness of the driver using, as the drowsiness estimation feature amount, the feature amount excluding the feature amount calculated due to the driver taking the noise factor action on the basis of the captured image acquired from the imaging device 2, and performing control to correct the drowsiness score on the basis of the drowsiness related information acquired on the basis of the captured image.

The processing circuit 1001 reads and executes the program stored in the memory 1005, thereby executing the functions of the sensing unit 11, the first noise factor detecting unit 12, the feature amount calculating unit 13, the drowsiness score calculating unit 14, the drowsiness estimation unit 15, the score correcting unit 17, and the third noise factor detecting unit 18. That is, the drowsiness estimation device 1b includes the memory 1005 for storing the program that results in execution of steps ST10 to ST50 and step ST70 of FIG. 7 described above when executed by the processing circuit 1001. It can also be said that the program stored in the memory 1005 causes a computer to execute a procedure or a method of the sensing unit 11, the first noise factor detecting unit 12, the feature amount calculating unit 13, the drowsiness score calculating unit 14, the drowsiness estimation unit 15, the score correcting unit 17, and the third noise factor detecting unit 18.

The drowsiness estimation device 1b includes an input interface device 1002 and an output interface device 1003 that perform wired communication or wireless communication with a device such as the imaging device 2.

In the third embodiment described above, the drowsiness estimation device 1b is an in-vehicle device mounted on a vehicle, and the sensing unit 11, the first noise factor detecting unit 12, the feature amount calculating unit 13, the drowsiness score calculating unit 14, the drowsiness estimation unit 15, the score correcting unit 17, and the third noise factor detecting unit 18 are included in the drowsiness estimation device 1b.

The embodiment is not limited thereto, and a drowsiness estimation system may be configured by an in-vehicle device and a server in which a part of the sensing unit 11, the first noise factor detecting unit 12, the feature amount calculating unit 13, the drowsiness score calculating unit 14, the drowsiness estimation unit 15, the score correcting unit 17, and the third noise factor detecting unit 18 is mounted on the in-vehicle device of the vehicle and the others are provided in the server connected to the in-vehicle device via a network.

Alternatively, the sensing unit 11, the first noise factor detecting unit 12, the feature amount calculating unit 13, the drowsiness score calculating unit 14, the drowsiness estimation unit 15, the score correcting unit 17, and the third noise factor detecting unit 18 may all be included in the server.

Further, in the third embodiment described above, the subject is a driver of the vehicle as an example, but this is merely an example. The subject may be an occupant other than the driver of the vehicle. Furthermore, the subject may be an occupant including a driver of a mobile object other than a vehicle, such as a bus, a train, or an airplane. The drowsiness estimation device 1b according to the second embodiment can be applied as a drowsiness estimation device that estimates drowsiness of an occupant of a mobile object other than the vehicle.

As described above, according to the third embodiment, the drowsiness estimation device 1b includes the sensing unit 11 to acquire drowsiness related information indicating a state related to drowsiness of an occupant of a mobile object for each of frames on the basis of frames of a captured image obtained by capturing a face of the occupant, the first noise factor detecting unit 12 to detect a noise factor action that is an action accompanied by eye movement and is similar to an action of the occupant caused by drowsiness on the basis of the drowsiness related information acquired by the sensing unit 11, the feature amount calculating unit 13 to calculate a drowsiness estimation feature amount for estimating the drowsiness of the occupant on the basis of post-noise factor removal drowsiness related information after excluding the drowsiness related information that is a source of detection of the noise factor action by the first noise factor detecting unit 12 from the drowsiness related information acquired by the sensing unit 11, the drowsiness score calculating unit 14 to calculate a drowsiness score using the drowsiness estimation feature amount calculated by the feature amount calculating unit 13 and the drowsiness estimation unit 15 to estimate the drowsiness of the occupant on the basis of the drowsiness score calculated by the drowsiness score calculating unit 14. Therefore, in estimating drowsiness of the occupant of the mobile object, the drowsiness estimation device 1b can prevent a decrease in estimation accuracy of the drowsiness of the occupant due to that the occupant takes an action in which a feature similar to a feature when the drowsiness occurs is observed.

Furthermore, the drowsiness estimation device 1b includes the third noise factor detecting unit 18 to detect, on the basis of the drowsiness related information acquired by the sensing unit 11, occurrence of noise factor sensing that is an event in which it is estimated that the drowsiness related information is erroneously acquired due to that the sensing unit 11 erroneously detects the state related to the drowsiness of the occupant, and the score correcting unit 17 to correct the drowsiness score calculated by the drowsiness score calculating unit 14 when the third noise factor detecting unit 18 detects the occurrence of the noise factor sensing, in which the drowsiness estimation unit 15 is configured to estimate the drowsiness of the occupant on the basis of a post-correction drowsiness score after correction by the score correcting unit 17 when the score correcting unit 17 corrects the drowsiness score calculated by the drowsiness score calculating unit 14. Therefore, the drowsiness estimation device 1b can prevent over-estimation of drowsiness of the occupant.

Note that the configuration of the drowsiness estimation device may be a combination of the configurations of the drowsiness estimation device 1 according to the first embodiment described above, the drowsiness estimation device 1a according to the second embodiment, and the drowsiness estimation device 1b according to the third embodiment.

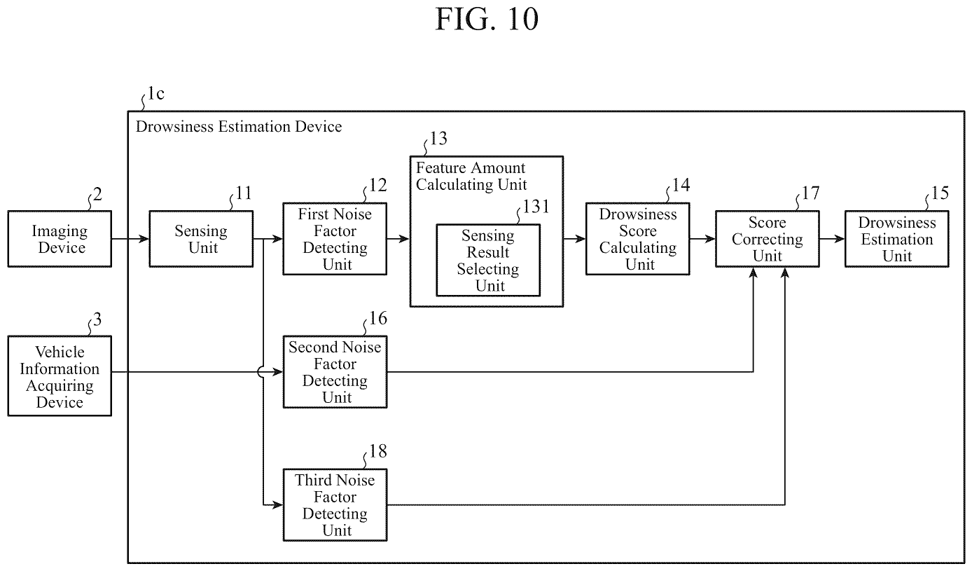

FIG. 10 is a diagram illustrating a configuration example of a drowsiness estimation device 1c obtained by combining the configurations of the drowsiness estimation device 1 according to the first embodiment, the drowsiness estimation device 1a according to the second embodiment, and the drowsiness estimation device 1b according to the third embodiment.

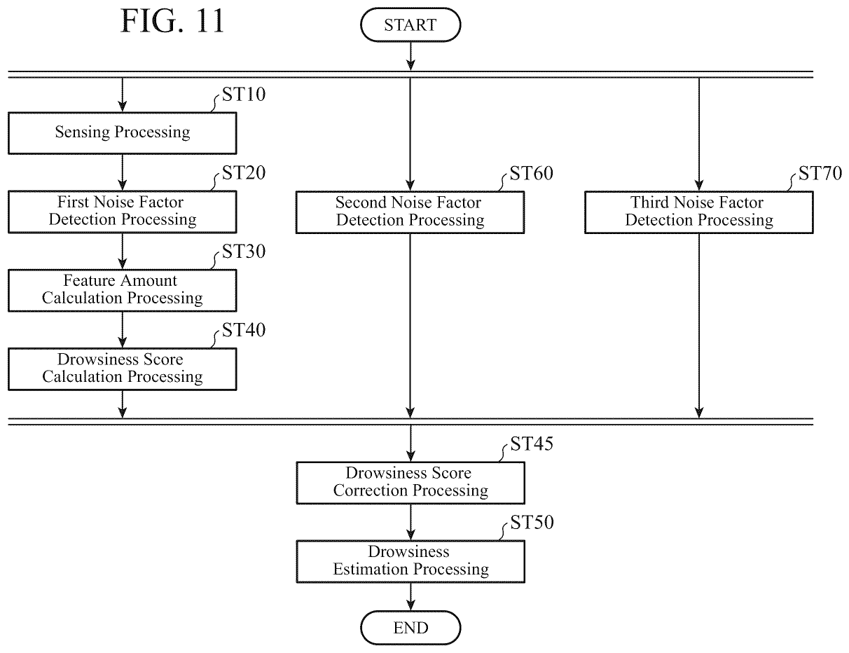

FIG. 11 is a flowchart for describing an operation of the drowsiness estimation device 1c in which the configurations of the drowsiness estimation device 1 according to the first embodiment, the drowsiness estimation device 1a according to the second embodiment, and the drowsiness estimation device 1b according to the third embodiment are combined.

As described above, in addition to the sensing unit 11 to acquire the drowsiness related information indicating the state related to the drowsiness of the occupant (for example, the driver) for each frame on the basis of the frame of the captured image obtained by capturing the face of the occupant (for example, the driver) of the mobile object (for example, the vehicle), the first noise factor detecting unit 12 to detect the noise factor action by the occupant on the basis of the drowsiness related information acquired by the sensing unit 11, the feature amount calculating unit 13 to calculate the drowsiness estimation feature amount for estimating the drowsiness of the occupant on the basis of
the post-noise factor removal drowsiness related information after excluding the drowsiness related information from which the first noise factor detecting unit 12 has detected the noise factor action from the drowsiness related information acquired by the sensing unit 11, the drowsiness score calculating unit 14 to calculate the drowsiness score using the drowsiness estimation feature amount calculated by the feature amount calculating unit 13, and the drowsiness estimation unit 15 to estimate the drowsiness of the occupant on the basis of the drowsiness score calculated by the drowsiness score calculating unit 14, the drowsiness estimation device 1c includes: the second noise factor detecting unit 16 to detect a noise factor traveling state that is assumed to be unlikely to cause the drowsiness in the occupant (for example, the driver) on the basis of the mobile object information (vehicle information) related to the mobile object (for example, the vehicle); the third noise factor detecting unit 18 to detect, on the basis of the drowsiness related information acquired by the sensing unit 11, occurrence of noise factor sensing that is an event in which it is estimated that the drowsiness related information is erroneously acquired due to that the sensing unit 11 erroneously detects the state related to the drowsiness of the occupant; and the score correcting unit 17 to correct the drowsiness score calculated by the drowsiness score calculating unit 14 on the basis of the noise factor traveling state or the occurrence of the noise factor sensing when the second noise factor detecting unit 16 detects the noise factor traveling state or the third noise factor detecting unit 18 detects the occurrence of noise factor sensing, in which the drowsiness estimation unit 15 estimates the drowsiness of the occupant on the basis of a post-correction drowsiness score after correction by the score correcting unit 17.

Thus, in estimating the drowsiness of the occupant, the drowsiness estimation device 1c can prevent the estimation accuracy of the drowsiness of the occupant from deteriorating due to the occupant taking the noise factor action, and more specifically, in estimating the drowsiness of the occupant, the drowsiness estimation device 1c can prevent the estimation accuracy of the drowsiness of the occupant from deteriorating due to use of a feature amount that becomes noise for estimation of the drowsiness of the occupant, perform accurate drowsiness estimation, and prevent over-estimation of the drowsiness of the occupant.

Note that, in the drowsiness estimation device 1c, a rule in consideration of both the noise factor traveling state and the occurrence of the noise factor sensing is set as a score correction rule used when the score correcting unit 17 corrects the drowsiness score.

For example, as the score correction rule, a rule is set such that “when the noise factor traveling state is detected, the drowsiness score is multiplied by “0.5”, and when the occurrence of the noise factor sensing is detected, the drowsiness score is multiplied by “0.5”. When both the noise factor traveling state and the occurrence of the noise factor sensing are detected, a rule is set such that the drowsiness score is corrected with a value obtained by multiplying a multiplier corresponding to the noise factor traveling state (that is, “0.5”) by a multiplier corresponding to the occurrence of the noise factor sensing (that is, “0.5”)”.

Further, for example, as the score correction rule, a rule may be set such that “when the noise factor traveling state is detected, the drowsiness score is multiplied by “0.5”, and when the occurrence of the noise factor sensing is detected, the drowsiness score is multiplied by “0.5”. When both the noise factor traveling state and the occurrence of the noise factor sensing are detected, a rule may be set such that the drowsiness score is corrected in accordance with a weight set for the noise factor traveling state and the noise factor sensing”.

In each case of what noise factor traveling state and noise factor sensing, what weight is given is determined in advance by the administrator or the like.

Thus, the drowsiness estimation device 1c can further enhance the effect of correcting the drowsiness score.

Note that free combinations of the individual embodiments, modifications of any components of the individual embodiments, or omissions of any components in the individual embodiments are possible.

### INDUSTRIAL APPLICABILITY

In estimating drowsiness of an occupant of a mobile object, a drowsiness estimation device of the present disclosure can prevent a decrease in estimation accuracy of the drowsiness of the occupant due to that the occupant takes an action in which a feature similar to a feature when the drowsiness occurs is observed.

### REFERENCE SIGNS LIST

1, 1a, 1b, 1c: drowsiness estimation device, 2: imaging device, 3: vehicle information acquiring device, 11: sensing unit, 12: first noise factor detecting unit, 13: feature amount calculating unit, 131: sensing result selecting unit, 14: drowsiness score calculating unit, 15: drowsiness estimation unit, 16: second noise factor detecting unit, 17: score correcting unit, 18: third noise factor detecting unit, 1001: processing circuit, 1002: input interface device, 1003: output interface device, 1004: processor, 1005: memory

1. A drowsiness estimation device comprising:
a processor; and
a memory storing a program, upon executed by the processor, to perform a process:
to acquire drowsiness related information indicating a state related to drowsiness of an occupant of a mobile object for each of frames on a basis of frames of a captured image obtained by capturing a face of the occupant;
to detect a noise factor action that is an action accompanied by eye movement and is similar to an action of the occupant caused by drowsiness on a basis of the drowsiness related information acquired;
to calculate a drowsiness estimation feature amount for estimating the drowsiness of the occupant on a basis of post-noise factor removal drowsiness related information after excluding the drowsiness related information that is a source of detection of the noise factor action from the drowsiness related information acquired;
to calculate a drowsiness score using the drowsiness estimation feature amount calculated; and
to estimate the drowsiness of the occupant on a basis of the drowsiness score calculated.

2. The drowsiness estimation device according to claim 1, wherein
the process
includes to attach an exclusion target flag to the drowsiness related information that is a source of detection of the noise factor action, as the drowsiness related information to be an exclusion target in calculation of the drowsiness estimation feature amount, among the drowsiness related information acquired on a basis of noise factor action information related to the noise factor action detected, and
specifies the post-noise factor removal drowsiness related information by excluding the drowsiness related information to which the exclusion target flag is attached from the drowsiness related information acquired, and calculates the drowsiness estimation feature amount on a basis of the specified post-noise factor removal drowsiness related information.

3. The drowsiness estimation device according to claim 1, wherein
the process
includes to select, as the post-noise factor removal drowsiness related information, the drowsiness related information after excluding the drowsiness related information that is a source of detection of the noise factor action from among the drowsiness related information acquired on a basis of noise factor action information related to the noise factor action detected, and
calculates the drowsiness estimation feature amount on a basis of the post-noise factor removal drowsiness related information selected.

4. The drowsiness estimation device according to claim 1, the process comprising:
to detect a noise factor traveling state on a basis of mobile object information related to the mobile object, the noise factor traveling state being a traveling state of the mobile object that is assumed to be unlikely to cause the drowsiness in the occupant; and
to correct the drowsiness score calculated when the process detects the noise factor traveling state, wherein
the process estimates the drowsiness of the occupant on a basis of a post-correction drowsiness score after correction when the process corrects the drowsiness score calculated.

5. The drowsiness estimation device according to claim 4, wherein
the noise factor traveling state includes a state in which the mobile object is traveling at a low speed, a state in which a blinker use frequency is high while the mobile object is traveling, a state in which a frequency of use of a brake in the mobile object is high, or a state in which a change in a steering wheel angle of the mobile object is large.

6. The drowsiness estimation device according to claim 1, the process comprising:
to detect, on a basis of the drowsiness related information acquired, occurrence of noise factor sensing that is an event in which it is estimated that the drowsiness related information is erroneously acquired due to that the process erroneously detects the state related to the drowsiness of the occupant; and
to correct the drowsiness score calculated when the process detects the occurrence of the noise factor sensing, wherein
the process estimates the drowsiness of the occupant on a basis of a post-correction drowsiness score after correction unit when the process corrects the drowsiness score calculated.

7. The drowsiness estimation device according to claim 6, wherein
the process detects the occurrence of the noise factor sensing when the drowsiness related information having a possibility to be determined as a long continuous eye closing state is acquired, or when the drowsiness related information having a possibility to be determined to have a large number of blinks is acquired.

8. The drowsiness estimation device according to claim 1, the process comprising:
to detect a noise factor traveling state on a basis of mobile object information related to the mobile object, the noise factor traveling state being a traveling state of the mobile object that is assumed to be unlikely to cause the drowsiness in the occupant;
to detect, on a basis of the drowsiness related information acquired, occurrence of noise factor sensing that is an event in which it is estimated that the drowsiness related information is erroneously acquired due to that the process sensing erroneously detects the state related to the drowsiness of the occupant; and
to correct the drowsiness score calculated on a basis of the noise factor traveling state or the occurrence of the noise factor sensing when the process detects the noise factor traveling state or the process detects the occurrence of the noise factor sensing, wherein
the process estimates the drowsiness of the occupant on a basis of a post-correction drowsiness score after correction.

9. The drowsiness estimation device according to claim 8, wherein
when the process detects the noise factor traveling state and the process detects the occurrence of the noise factor sensing, the process corrects the drowsiness score in accordance with a weight set for the noise factor traveling state and the noise factor sensing.

10. The drowsiness estimation device according to claim 1, wherein
the occupant of the mobile object is a driver of a vehicle.

11. A drowsiness estimation method comprising:
acquiring drowsiness related information indicating a state related to drowsiness of an occupant of a mobile object for each of frames on a basis of frames of a captured image obtained by capturing a face of the occupant;
detecting a noise factor action that is an action accompanied by eye movement and is similar to an action of the occupant caused by drowsiness on a basis of the drowsiness related information acquired;
calculating a drowsiness estimation feature amount for estimating the drowsiness of the occupant on a basis of post-noise factor removal drowsiness related information after excluding the drowsiness related information that is a source of detection of the noise factor action from the drowsiness related information acquired;
calculating drowsiness score using the drowsiness estimation feature amount calculated; and
estimating the drowsiness of the occupant on a basis of the drowsiness score calculated.
